(19) United States
(12) Patent Application Publication
(10) Pub. No.: US 2002/0119995 A1
(43) Pub. Date: Aug. 29, 2002

(54) Inverse thiazolylamide derivatives

(76) Inventors: Martin Hendrix, Koln (DE); Gerald Kleymann, Wuppertal (DE); Ulrich Betz, Wuppertal (DE); Judith Baumeister, Wuppertal (DE); Wolfgang Bender, Wuppertal (DE); Peter Eckenberg, Wuppertal (DE); Rudiger Fischer, Pulheim (DE); Gabriele Handke, Wulfrath (DE); Kerstin Henninger, Wuppertal (DE); Axel Jensen, Velbert (DE); Jorg Keldenich, Wuppertal (DE); Udo Schneider, Leverkusen (DE); Olaf Weber, Woodbridge, CT (US)

Correspondence Address: Jeffrey M. Greeman, Vice President, Patents and Licensing, Bayer Corporation, 400 Morgan Lane, West Haven, CT, 06516, US

(21) Appl. No.: 09/918,994

(22) Filed: Jul. 31, 2001

(30) Foreign Application Priority Data
Aug. 4, 2000 (DE) 10038022.0

(51) Int. Cl. C07D 417/02; C07D 277/36; A61K 31/454; A61K 31/426; A61K 31/427

(52) U.S. Cl. 514/326; 548/187; 546/209; 514/369

(57) ABSTRACT

The present invention relates to novel compounds, i.e. inverse thiazolylamide derivatives, to processes for their preparation and to their use as medicaments, in particular as antiviral medicaments.

The present invention relates to novel compounds, i.e. inverse thiazolylamide derivatives, to processes for their preparation and to their use as medicaments, in particular as antiviral medicaments.

2-Methyl-thiazole-5-sulphonamides are disclosed in the publications R. J. Cremlyn et al., J. Heterocycl. Chem.; EN; 18; 1981; 997-1006 and R. Bellemin et al., J. Heterocycl. Chem.; EN; 21; 1984; 1017-1021 and R. E. Olson et al.; JMCMAR; J. Med. Chem.; EN; 42; 7; 1999; 1178-1192.

WO 97/24343 and WO 99/42455 relate to phenylthiazole derivatives having anti-herpes-virus properties.

WO 99/47507 relates to 1,3,4-thiadiazole derivatives having anti-herpes-virus properties.

The present invention relates to novel compounds which are thiazolylamide derivatives of the general formula (I):

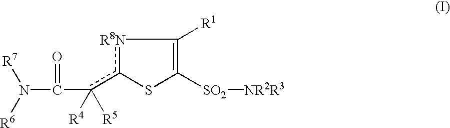

in which

R¹ represents hydrogen, halogen, (C₁-C₆)-alkyl, (C₁—C₆)-alkoxy, amino-(C₁-C₆)-alkyl or halogeno-(C₁-C₆)-alkyl,

R² and R³ are identical or different and represent hydrogen or represent (C₁-C₆)-alkyl which is optionally substituted by 1 to 3 substituents selected from the group consisting of (C₃-C₆)-cycloalkyl, (C₁-C₆)-alkoxy, halogen, hydroxyl, amino and (C₆-C₁₀)-aryl which for its part may be substituted by hydroxyl or (C₁-C₆)-alkoxy, or

R² and R³ together with the nitrogen atom form a 5- or 6-membered saturated heterocycle which may optionally also contain an oxygen atom,

R⁴ represents hydrogen, (C₁-C₆)-acyl, (C₂-C₆)-alkenyl, (C₃-C₈)-cycloalkyl or (C₁-C₆)-alkoxycarbonyl, or

R⁴ represents (C₁-C₆)-alkyl which may optionally be substituted by 1 to 3 substituents selected from the group consisting of halogen, hydroxyl, (C₁-C₆)-acyl, (C₁-C₆)-alkoxy, —(OCH₂CH₂)ₙOCH₂CH₃, where n is 0 or 1, phenoxy, (C₆-C₁₀)-aryl and —NR¹³R¹⁴,

where

R¹³ and R¹⁴ are identical or different and represent hydrogen, (C₁-C₆)-acyl, (C₁-C₆)-alkyl, carbamoyl, mono- or di-(C₁-C₆)-alkylamino-(C₁-C₆)-alkyl, mono- or di-(C₁-C₆)-alkylaminocarbonyl, (C₆-C₁₀)-aryl or (C₁-C₆)-alkoxycarbonyl, or

R¹³ and R¹⁴ together with the nitrogen atom form a 5- or 6-membered saturated heterocycle which may optionally contain a further heteroatom from the group consisting of S and O or a radical of the formula —NR¹⁵ and which may be substituted by oxo,

where

R¹⁵ represents hydrogen or (C₁-C₄)-alkyl,

R⁵ represents no substituent, hydrogen, (C₁-C₆)-alkyl or (C₁-C₆)-alkanoyl,

R⁶ represents phenyl which may optionally be substituted by one to three substituents selected from the group consisting of

halogen,

(C₆-C₁₀)-aryl which may optionally be substituted by 1 to 3 substituents selected from the group consisting of (C₁-C₆)-alkanoyl, (C₁-C₆)-alkoxy, (C₁-C₆)-alkyl, halogen, (C₁-C₆)-alkoxycarbonyl, nitro, halogeno-(C₁-C₆)-alkyl, halogeno-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulphoxy, (C₁-C₆)-alkylsulphonyl, tri-(C₁-C₆)-alkylsilyloxy, a 3- to 8-membered saturated or unsaturated non-aromatic mono- or bicyclic heterocycle having up to 3 heteroatoms from the group consisting of S, N and O, which is optionally attached via a nitrogen atom, and cyano,

(C₁-C₆)-alkoxycarbonyl,

(C₁-C₆)-alkylthio,

hydroxyl,

carboxyl,

partially fluorinated (C₁-C₆)-alkoxy having up to 6 fluorine atoms,

(C₁-C₆)-alkyl which may optionally be substituted by a radical of the formula

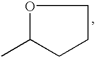

an aromatic heterocycle having up to 3 hetero atoms from the group consisting of S, N and O, which is optionally attached via a nitrogen atom and may optionally be substituted by 1 to 3 substituents selected from the group consisting of (C_{1 -C}₆)-alkanoyl, (C₁-C₆)-alkoxy, (C₁-C₆)-alkyl, halogen, (C₁-C₆)-alkoxycarbonyl, nitro, halogeno-(C₁-C₆)-alkyl, halogeno-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulphoxy, (C₁-C₆)-alkylsulphonyl, a 3- to 8-membered saturated or unsaturated non-aromatic mono- or bicyclic heterocycle having up to 3 heteroatoms from the group consisting of S, N and O, which is optionally attached via a nitrogen atom, and cyano,

a 3- to 8-membered saturated or unsaturated non-aromatic mono- or bicyclic heterocycle having up to 3 heteroatoms from the group consisting of S, N and O, which is optionally attached via a nitrogen atom and which may optionally be substituted by 1 to 3 substituents selected from the group consisting of oxo, halogen, hydroxyl, (C₁-C₆)-alkoxycarbonyl, (C₁-C₆)-alkoxycarbonylamino, (C₁-C₆)-alkyl, halogeno-(C₁-C₆)-alkyl and hydroxy-(C₁-C₆)-alkyl,

and groups of the formulae

—OR¹⁹,

—NR²⁰R²¹ or —CO—NR²²R²³,

in which

R¹⁹ is phenyl which for its part is optionally substituted by a group of the formula —NR²⁴R₂₅,

in which

R²⁴ and R²⁵ are identical or different and represent hydrogen, (C₁-C₆)-alkyl or (C₁-C₆)-acyl,

or

R¹⁹ represents (C₁-C₆)-alkyl which is optionally mono- to trisubstituted by hydroxyl and/or halogen,

R²⁰ and R²¹ are identical or different and hydrogen, carbamoyl, mono- or di-(C₁-C₆)-alkylaminocarbonyl, phenyl, (C₁-C₆)-acyl or (C₁-C₆)-alkyl, where the (C₁-C₆)-alkyl mentioned above is optionally substituted by (C₁-C₆)-alkoxy, (C₁-C₆)-acyl, by phenyl or by a 5- or 6-membered aromatic heterocycle having up to 3 heteroatoms from the group consisting of S, N and O, where the phenyl mentioned above and the aromatic heterocycle mentioned above are optionally mono- to trisubstituted by identical or different substituents from the group consisting of halogen and hydroxyl, and

R²² and R²³ are identical or different and represent hydrogen or (C₁-C₆)-alkyl,

R⁷ represents hydrogen, (C₁-C₆)-alkyl or (C₁-C₆)-alkanoyl,

R⁸ represents no substituent, hydrogen or (C₁-C₆)-alkyl, and

[symbol] represents a single or double bond,

and their salts.

Physiologically acceptable salts of the compounds according to the invention can be, for example, salts of the substances according to the invention with mineral acids, carboxylic acids or sulphonic acids. Particular preference is given, for example, to salts with hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, citric acid, fumaric acid, maleic acid, or benzoic acid.

Salts which may furthermore be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine, 1-ephenamine or methylpiperidine.

Depending on the substitution pattern, the compounds according to the invention can exist in stereoisomeric forms which either behave as image and mirror image (enantiomers), or which do not behave as image and mirror image (diastereomers). The invention relates both to the enantiomers or diastereomers and their respective mixtures. Like the diastereomers, the racemic forms can be separated into the stereoisomerically uniform components in a known manner.

The scope of the invention includes those compounds which are only converted into the actual active compounds of the formula (I) once inside the body (“prodrugs”).

In the context of the present invention, in the molecular moiety shown,

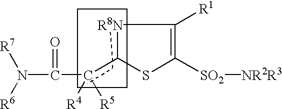

[symbol] means that a single and a double bond are present along the endo- and the exocyclic bond. In some cases, these compounds are in an isomeric relationship with respect to the bond and can be converted into one another. The normal valencies of the atoms involved are not exceeded, i.e., for example, the ring nitrogen printed in bold does not carry a substituent R⁸ if the double bond is endocyclic and the single bond is exocyclic. In turn, the carbon atom printed in bold does not carry a substituent R⁵ if the double bond is exocyclic and the single bond is endocyclic. These forms can be represented as follows:

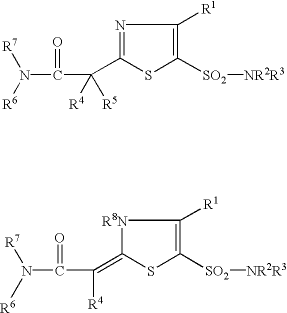

(C₁-C₆)-Alkyl advantageously represents a straight-chain or branched alkyl radical having from 1 to 6 carbon atoms. Preference is given to a straight-chain or branched alkyl radical having 1 to 4 carbon atoms (C₁-C₄). Examples which may be mentioned are: methyl, ethyl, n-propyl, isopropyl, n-butyl, sec-butyl, tert-butyl, n-pentyl and n-hexyl. Particular preference is given to a straight-chain or branched alkyl radical having 1 to 3 carbon atoms ((C₁-C₃)-alkyl).

Halogeno-(C₁-C₆)-alkyl advantageously represents a (C₁-C₆)-alkyl group which can be defined as above and which has 1 to 3 halogen atoms, namely F, Cl, Br and/or I, preferably chlorine or fluorine, as substituents; examples which may be mentioned are trifluoromethyl, fluoromethyl, etc.

Hydroxy-(C₁-C₆)-alkyl advantageously represents a (C₁-C₆)-alkyl group which can be defined as above and which has 1 to 3 hydroxyl groups as substituents; examples which may be mentioned are hydroxymethyl etc.

(C₂-C₆)-Alkenyl in the context of the invention advantageously represents a straight-chain or branched alkenyl radical having 2 to 6 carbon atoms. Examples which may be mentioned are: ethenyl, n-prop-2-en-1-yl and n-but-2-en-1-yl. Preference is given to a straight-chain or branched alkenyl radical having 2 to 4 carbon atoms.

(C₁-C₆)-Alkoxy advantageously represents a straight-chain or branched alkoxy radical having 1 to 6 carbon atoms. Preference is given to a straight-chain or branched alkoxy radical having 1 to 4 carbon atoms (C₁-C₄). Examples which may be mentioned are: methoxy, ethoxy, n-propoxy, isopropoxy, tert-butoxy, n-pentoxy and n-hexoxy. Particular preference is given to a straight-chain or branched alkoxy radical having 1 to 3 carbon atoms (C₁-C₃).

Halogeno-(C₁-C₆)-alkoxy advantageously represents mono- or polyhalogenated (C₁-C₆)-alkoxy. With respect to the (C₁-C₆)-alkoxy moiety and the definition of halogen, reference is made to the above definition. Halogeno-(C₁-C₆)-alkoxy includes, for example, partially mono- or polychlorinated and/or -fluorinated or -perfluorinated (C₁-C₆)-alkoxy, such as trifluoromethoxy, fluoromethoxy, chloromethoxy, pentafluoroethoxy, trifluoromethylmethoxy, etc.

Partially fluorinated (C₁-C₆)-alkoxy having up to 6 fluorine atoms advantageously represents a straight-chain or branched alkoxy radical having 1 to 6 carbon atoms which may be substituted by 1 to 6, preferably 1 to 4, more preferably 1 to 3, fluorine atoms. Preference is given to a straight-chain or branched alkoxy radical having 1 to 4 carbon atoms and 1 to 4 fluorine atoms. Examples which may be mentioned are: methoxy, ethoxy, n-propoxy, isopropoxy, tert-butoxy, n-pentoxy and n-hexoxy, which in each case have 1 to 4 fluorine atoms. Particular preference is given to (1,3-difluoroprop-2-yl)-oxy and 1,1,2,2-tetrafluorethoxy.

(C₁-C₆)-Alkylthio advantageously represents a straight-chain or branched alkylthio radical having 1 to 6 carbon atoms. Preference is given to a straight-chain or branched alkylthio radical having 1 to 4 carbon atoms (C₁-C₄). Examples which may be mentioned are: methylthio, ethylthio, n-propylthio, isopropylthio, tert-butylthio, n-pentylthio and n-hexylthio. Particular preference is given to a straight-chain or branched alkylthio radical having 1 to 3 carbon atoms (C₁-C₃)-alkylthio.

(C₁-C₆)-Alkoxycarbonyl advantageously represents a straight-chain or branched alkoxycarbonyl radical having 1 to 6 carbon atoms. Preference is given to a straight-chain or branched alkoxycarbonyl radical having 1 to 4 carbon atoms (C₁-C₄). Examples which may be mentioned are: methoxycarbonyl, ethoxycarbonyl, n-propoxycarbonyl, isopropoxycarbonyl and tert-butoxycarbonyl. Particular preference is given to a straight-chain or branched alkoxycarbonyl radical having 1 to 4 carbon atoms (C₁-C₄).

Mono- or di-(C₁-C₆)-alkylaminocarbonyl in the context of the invention advantageously represents a carbamoyl group (H₂N—CO—), in which one or both hydrogen atoms are replaced by a (C₁-C₆)-alkyl group. With respect to the definition of the (C₁-C₆)-alkyl group, reference is made to the above explanation of (C₁-C₆)-alkyl. Examples which may be mentioned are methylaminocarbonyl, dimethylaminocarbonyl, etc.

Mono- or di-(C₁-C₆)-acylamino in the context of the invention advantageously represents an amino group (H₂N—) in which one or both hydrogen atoms are replaced by a (C₁-C₆)-acyl group. With respect to the definition of the (C₁-C₆)-acyl group, reference is made to the above explanation of (C₁-C₆)-acyl. An example which may be mentioned is (C₁-C₆)-alkanoyl, as mentioned in the definition of (C₁-C₆)-acyl.

(C₁-C₆)-Alkylsulphoxy advantageously represents a (C₁-C₆)-alkyl-S(═O) group, where, with respect to the (C₁-C₆)-alkyl group, reference can be made to the relevant definition above.

(C₁-C₆)-Alkylsulphonyl advantageously represents a (C₁-C₆)-alkyl-SO₂ group where, with respect to the (C₁-C₆)-alkyl group, reference can be made to the relevant definition above.

(C₆-C₁₀)-Aryl generally represents an aromatic radical having 6 to 10 carbon atoms. Preferred aryl radicals are phenyl and naphthyl.

(C₁-C₆)-Acyl in the context of the invention advantageously represents a straight-chain or branched acyl radical having 1 to 6 carbon atoms. Examples which may be mentioned are: formyl, acetyl, ethanoyl, propanoyl, isopropanoyl, butanoyl, isobutanoyl and pentanoyl. Preference is given to a straight-chain or branched acyl radical having 1 to 4 carbon atoms. Particular preference is given to acetyl and ethanoyl.

(C₃-C₈)-Cycloalkyl in the context of the invention represents cyclopropyl, cyclopentyl, cyclobutyl, cyclohexyl, cycloheptyl or cyclooctyl. Cyclopropyl, cyclopentyl and cyclohexyl may be mentioned as being preferred. The meaning of (C₃-C₆)-cycloalkyl is correspondingly advantageously cyclopropyl, cyclopentyl, cyclobutyl, cyclohexyl.

Halogen in the context of the invention generally represents fluorine, chlorine, bromine and iodine. Preference is given to fluorine, chlorine and bromine. Particular preference is given to fluorine and chlorine.

(C₁-C₆)-Alkanoyl in the context of the invention represents formyl and (C₁-C₅)-alkylcarbonyl groups, where (C₁-C₅)-alkyl may be a straight-chain or branched-chain alkyl group having 1 to 5 carbon atoms, for example acetyl, propionyl, butyryl, pentanoyl.

A 5- or 6-membered aromatic heterocycle having up to 3 heteroatoms from the group consisting of S, O and N represents, for example, pyridyl, pyrimidyl, thienyl, furyl, pyrrolyl, thiazolyl, N-triazolyl, oxazolyl or imidazolyl. Preference is given to pyridyl, furyl, thiazolyl and N-triazolyl.

A 5- or 6-membered aromatic benzo-fused heterocycle having up to 3 heteroatoms from the group consisting of S, O and N represents, for example, benzimidazolyl.

A 5- or 6-membered saturated heterocycle attached via a nitrogen atom, which can be formed from two substituent groups together with the nitrogen atom to which they are attached, and which may optionally contain a further heteroatom from the group consisting of S and O or a radical of the formula —NR¹⁵, in which R¹⁵ is as defined above, generally represents, in the context of the invention, morpholinyl, piperidinyl, piperazinyl, methylpiperazinyl, thiomorpholinyl or pyrrolidinyl. Particular preference is given to morpholinyl, piperidinyl, pyrrolidinyl and thiomorpholinyl.

A 3- to 8-membered saturated or unsaturated non-aromatic heterocycle which is optionally attached via a nitrogen atom and which has up to 3 heteroatoms from the group consisting of S, N and O includes, for example, the abovementioned 5- or 6-membered saturated heterocycles which are attached via a nitrogen atom, and also 3-, 7- and 8-membered heterocycles, such as, for example, aziridines (for example 1-azacyclopropan-1-yl), azetidines (for example 1-azacyclobutan-1-yl) and azepines (for example 1-azepan-1-yl). The unsaturated representatives may contain 1 or 2 double bonds in the ring.

In a preferred embodiment, the invention relates to compounds of the general formula (I) in which R₁ represents hydrogen or (C₁-C₆)-alkyl. Particular preference is given to methyl.

In a further preferred embodiment, the invention relates to compounds of the general formula (I) in which R² and R³ each independently represent hydrogen or (C₁-C₆)-alkyl.

In a further preferred embodiment, the invention relates to compounds of the general formula (I) in which R⁴ represents hydrogen or (C₁-C₆)-alkyl. Particular preference is given to hydrogen and methyl.

In a further preferred embodiment, the invention relates to compounds of the general formula (I) in which R⁵ represents hydrogen.

In a further preferred embodiment, the invention relates to compounds of the general formula (I) in which R⁸ represents hydrogen or no substituent, in particular no substituent.

In a further preferred embodiment, the invention relates to compounds of the general formula (I) in which

R⁶ represents phenyl which may optionally be substituted by one to three substituents selected from the group consisting of

(C₆-C₁₀)-aryl which may optionally be substituted by 1 to 3 substituents selected from halogen, and

a 6-membered aromatic heterocycle having 1 heteroatom from the group consisting of S, N and O, which may optionally be substituted by 1 or 2 halogen atoms,

(C₁-C₆)-alkoxy.

In a further preferred embodiment, the invention relates to compounds of the general formula (I) in which the compounds have the following formula:

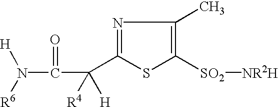

Particular preference is given to the compounds of Preparation Examples 1-3, 5 and 6 and to pharmaceutically acceptable salts thereof.

The invention furthermore relates to a process for preparing compounds of the general formula (I), characterized in that

compounds of the general formula (I′)

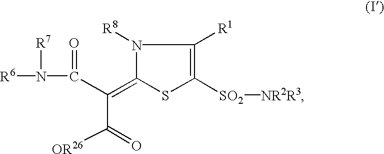

in which

R¹, R², R³, R⁶, R⁷ and R⁸ are as defined above and R²⁶ represents (C¹-C₆)-alkyl, are,

[A] if R⁴ is to be methyl, reacted with lithium aluminium hydride in inert solvents to give compounds of the general formula (I″)

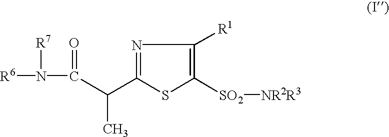

in which

R¹, R², R³, R⁶ and R⁷ are as defined above, or

[B] if R⁴ is to be hydrogen, reacted with lithium hydroxide in inert solvents to give compounds of the general formula (I′″)

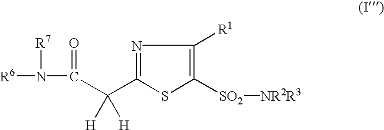

in which

R¹, R², R³, R⁶ and R⁷ are as defined above,

or

[C] if R⁴ is not to be hydrogen or methyl, reacted with compounds of the general formula (II)

Y-R⁴ (II)

in which Y represents a leaving group, such as, for example, triflate or halogen, preferably chlorine, and R⁴ is as defined above,

in inert solvents to give compounds of the general formula (III)

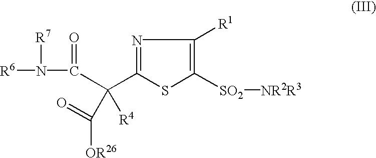

in which

R¹, R², R³, R⁴, R⁶, R⁷ and R²⁶ are as defined above,

and these are then decarboxylated to give compounds of the general formula (I ″″)

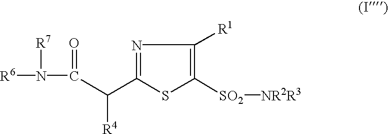

in which

R¹, R², R³, R⁴, R⁶ and R⁷ are as defined above,

and, if R⁵ is a substituent different from hydrogen,

compounds of the general formula (I″) or (I″″)

are reacted with compounds of the general formula (IV)

X-R⁵ (IV)

in which

X represents a leaving group, such as, for example, triflate or halogen, preferably chlorine, and R⁵ is as defined above, in inert solvents, if appropriate in the presence of a base and/or an auxiliary, to give compounds of the formula (I).

The processes [A], [B] and [C] according to the invention can be illustrated in an exemplary manner by the formula scheme below:

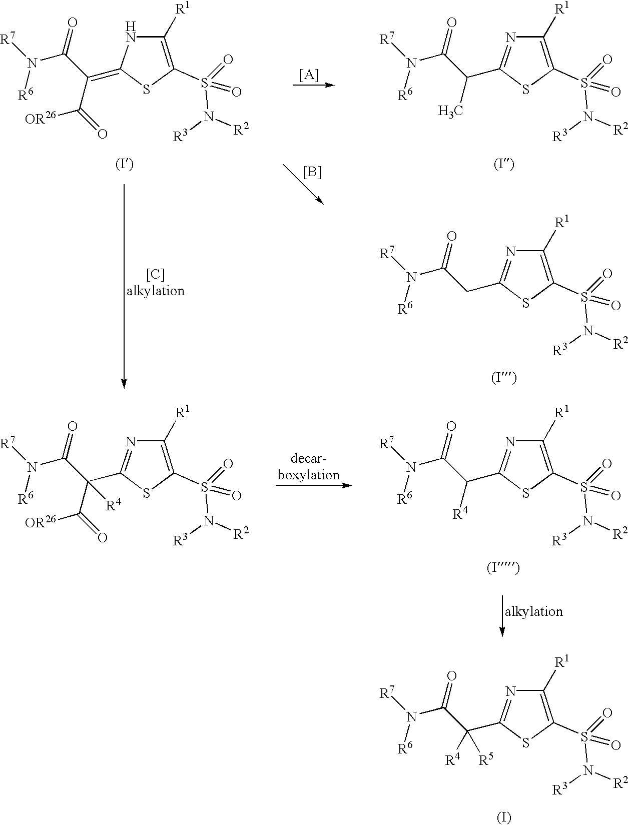

Suitable solvents for the processes [A], [B] and [C] are customary organic solvents which do not change under the reaction conditions. These preferably include ethers, such as diethyl ether, dioxane, tetrahydrofuran (THF), glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorobenzene, or ethyl acetate, dimethyl sulphoxide, dimethylformamide (DMF) or acetonitrile. It is also possible to use mixtures of the solvents mentioned. Preferences is given to THF.

The processes according to the invention are generally carried out in a temperature range from −50° C. to +100° C., preferably from −30° C. to +60° C.

The processes according to the invention are generally carried out at atmospheric pressure. However, it is also possible to carry out the processes at elevated pressure or at reduced pressure (for example in the range from 0.5 to 5 bar).

The compounds of the general formula (II) can be prepared, for example, by converting compounds of the general formula (V)

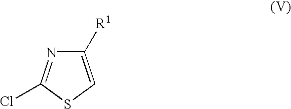

in which

R^{l} is as defined above,

by reaction with the system chlorosulphonic acid/SOCl₂ into the compounds of the general formula (VI)

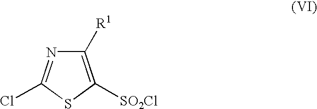

in which

R¹ is as defined above,

then, using amines of the general formula (VII)

HNR²R³ (VII)

in which

R² and R³ are as defined above,

in inert solvents, preparing the compounds of the general formula (VIII)

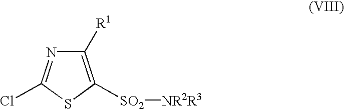

in which

R¹, R² and R³ are as defined above,

and, using compounds of the general formula (IX)

[R²⁶OC(O)]₂CH₂ (IX)

in which

R²⁶ represents (C₁-C₆)-alkyl,

preparing compounds of the general formula (X)

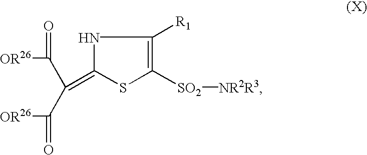

in which

R¹, R², R³ and R²⁶ are as defined above,

and, in a further step, carrying out a reaction with compounds of the general formula (XI)

R⁶R⁷NH (XI)

in which

R⁶ and R⁷ are as defined above, in inert solvents, to give compounds of the general formula (I′).

The reaction with chlorosulphonic acid/SO₂Cl is initially carried out at room temperature and then at the reflux temperature of the solvent in question.

The reaction is generally carried out under atmospheric pressure. However, it is also possible to carry out the process at elevated pressure or at reduced pressure (for example in the range from 0.5 to 5 bar).

Suitable solvents for the reaction with the amines of the general formula (VII) are alcohols, such as, for example methanol, ethanol, propanol and isopropanol. Preference is given to methanol.

The reaction with the amines of the general formula (VII) is initially carried out at room temperature and then at the reflux temperature of the alcohol in question.

The reaction is generally carried out at atmospheric pressure. However, it is also possible to carry out the process at elevated pressure or at reduced pressure (for example in the range from 0.5 to 5 bar).

Suitable solvents for the reaction with the compounds of the general formula (IX) are customary organic solvents which do not change under the reaction conditions. These preferably include hydrocarbons, such as benzene, toluene, xylene, hexane, cyclohexane or mineral oil fractions. It is also possible to use mixtures of the solvents mentioned. Preference is given to toluene.

Compounds of the general formula (IX) are known or can be prepared by customary methods.

The reaction is generally carried out at atmospheric pressure. However, it is also possible to carry out the process at elevated pressure or at reduced pressure (for example in the range from 0.5 to 5 bar).

The reaction with the compounds of the general formula (IX) is initially carried out at room temperature and then at the reflux temperature of the solvent in question.

Suitable solvents for the reaction with the compounds of the general formula (XI) are customary organic solvents which do not change under the reaction conditions. These preferably include hydrocarbons, such as benzene, toluene, xylene, hexane, cyclohexane or mineral oil fractions. It is also possible to use mixtures of the solvents mentioned. Preference is given to xylene.

The reaction with the compounds of the general formula (XI) is initially carried out at room temperature and then at the reflux temperature of the solvent in question.

Compounds of the general formula (XI) are known or can be prepared by customary methods.

The reaction with the compounds of the general formula (II), i.e. alkylating agents, acylating agents, etc., is advantageously carried out in dimethylformamide (DMF) or in another polar inert solvent, with addition of a suitable base, such as, for example, sodium hydride, an alkali metal alkoxide or lithium diethylamide.

The reaction is generally carried out at atmospheric pressure. However, it is also possible to carry out the process at elevated pressure or at reduced pressure (for example in the range from 0.5 to 5 bar).

Compounds of the general formula (II) are known or can be prepared by customary methods.

The invention furthermore relates to the compounds of the formula (X).

The invention furthermore relates to the compounds of the formula (I) for use as medicaments.

The invention furthermore relates to a pharmaceutical composition comprising a compound of the general formula (I) in a mixture with at least one pharmaceutically acceptable carrier or excipient.

The invention furthermore relates to the use of a compound of the general formula (I) for preparing a medicament, in particular a medicament for the treatment and/or prevention of viral infections, such as herpes viruses, in particular Herpes simplex viruses.

The invention furthermore relates to the use of [5-(aminosulphonyl)-1,3-thiazol-2-yl]propanamide and -acetamide derivatives for preparing medicaments, in particular for the use of the derivatives mentioned for preparing compositions for the treatment and/or prevention of viral infections in humans or animals, such as infections caused by herpes viruses, in particular by Herpes simplex viruses. Here, [5-(aminosulphonyl)-1,3-thiazol-2-yl]propanamide derivatives and [5-(aminosulphonyl)-1,3-thiazol-2-yl]acetamide derivatives are to be understood as meaning those compounds which are derived from [5-(aminosulphonyl)-1,3-thiazol-2-yl]propanamide and -acetamide by the substitution of one or more hydrogen atoms.

The compounds of the general formula (I) according to the invention exhibit an unforeseeable surprising spectrum of action. They exhibit an antiviral action against representatives of the herpesviridae group, particularly against Herpes simplex viruses (HSV). They are thus suitable for the treatment and prophylaxis of disorders which are caused by herpes viruses, in particular disorders which are caused by Herpes simplex viruses.

In vitro activity

Viruses and cells:

HSV (HSV-1 Walki, HSV-1F or HSV-2G) was cultivated on Vero cells (ATCC CCL-81) under the following conditions. The cells were grown in M199 medium (5% foetal calf serum, 2 mM glutamine, 100 IU/ml penicillin, 100 μg/ml streptomycin) in cell culture bottles at 37° C. and 5% CO₂. The cells were split twice per week, in each case 1:4. For the infection, the medium was removed, the cells were washed with Hank's solution, detached using 0.05% trypsin, 0.02% EDTA (Seromed L2143) and incubated at a density of 4×10⁵ cells per ml under the abovementioned conditions for 24 hours. The medium was then removed and the virus solution was added at an m.o.i. of <0.05 in a volume of 2 ml per 175 cm² of surface. The medium was incubated under the conditions mentioned for one hour and then made up to a volume of 50 ml per 175 cm² bottle. Three days after the infection, the cultures showed clear signs of a cytopathic effect. The virus was released by freezing (−80° C.) and thawing (37° C.) the cultures twice. Cell debris was removed by centrifugation (300 g, 10 min, 4° C.) and the supernatant was frozen down in aliquots at −80° C.

The virus titre was determined using a plaque assay. To this end, Vero cells were seeded in 24-well plates at a density of 4×10⁵ cells per well and, after 24 hours of incubation (37° C., 5% CO₂), infected with dilutions of the virus stock of from 10⁻² to 10¹² (100 μl of inoculum). One hour after the infection, the medium was removed and the cells were covered with 1 ml of overlay medium (0.5% methylcellulose, 0.22% sodium bicarbonate, 2 mM glutamine, 100 IU/ml penicillin, 100 μg/ml streptomycin, 5% foetal calf serum in MEM-Eagle medium with Earl's salt) and incubated for 3 days. The cells were then fixated using 4% formaline for 1hour, washed with water, stained with Giemsa (Merck) for 30 min and then washed and dried. Using a plaque viewer, the virus titre was determined. The virus stocks used for the experiments had a titre of 1×10⁶/ml- 1×10⁸/ml.

The anti-HSV action was determined in a screening test system in 96-well microtitre plates using various cell lines of neuronal, lymphoid and epithelial origin, such as, for example, Vero (kidney cell line of the green monkey), MEF (murine embryonal fibroblasts), HELF (human embryonal fibroblasts), NT2 (human neuronal cell line) or Jurkat (human lymphoid T-cell line). The effect of the substances on the spreading of the cytopathogenic effect was determined in comparison to the reference substance acyclovir-sodium (Zovirax^{R}), a clinically approved anti-herpes chemotherapeutic.

The substances (50 mM), dissolved in DMSO (dimethyl sulphoxide), are examined on microtitre plates (for example 96-well MTP) in final concentrations of 250-0.5 μM (micromolar) in two replications (4 substances/plate). In the case of potent substances, the dilutions are continued for several plates up to 0.5 pM (picomolar). Also examined are toxic and cytostatic effects of the substances. After an appropriate dilution of the substances (1:2) on the microtitre plate in medium, a suspension of cells (1×⁴ cells per well) such as, for example, of Vero cells in M199 (medium 199) with 5% foetal calf serum, 2 mM glutamine and optionally 100 IU/ml penicillin and 100 μg/ml streptomycin or of MEF cells in EMEM (Eagle's Minimum Essential Medium) with 10% foetal calf serum, 2 mM glutamine and optionally 100 IU/ml penicillin and 100 μg/ml streptomycin, or of HELF cells in EMEM with 10% foetal calf serum, 2 mM glutamine and optionally 100 IU/ml penicillin and 100 μg/ml streptomycin, or of NT2 and Jurkat cells in DMEM (4.5 mg/l glucose plus pyridoxin) with 10% foetal calf serum, 2 mM glutamine, 1 mM sodium pyruvate, non-essential amino acids and optionally 100 IU/ml penicillin and 100 μg/ml streptomycin, is added to each well and the cells in the relevant wells are infected with an appropriate amount of virus (HSV-1 F or HSV-2 G having an m.o.i. (multiplicity of infection) of 0.0025 for HELF, Vero and MEF cells and an m.o.i. of 0.1 for NT2 and Jurkat cells). The plates are then incubated at 37° C. in a CO₂ incubator (5% CO₂) for several days. After this time, the cell lawn of, for example, Vero cells in the substance-free virus controls, starting from 25 infection centres, is completely destroyed or lysed by the cytopathogenic effect of the HSV viruses (100% CPE). The plates are initially evaluated visually using a microscope and then analysed using a fluorescent dye. To this end, the cell supernatant of all wells of the MTP is aspirated and the wells are filled with 200 μl of PBS wash solution. The PBS is then aspirated and all the wells are filled with 200 μl of fluorescent dye solution (fluorescein diacetate, 10 μg/ml in PBS). After an incubation time of 30-90 min, the test plates are read in a fluorescence detector at an excitation wavelength of 485 nm and an emission wavelength of 538 nm.

The results for some compounds (Preparation Examples) are summarized in Table I below:

TABLE 1

| Example | IC₅₀ [μM] HSV-1 F/Vero | IC₅₀ [μM] HSV-2 G/Vero |
|---|---|---|
| Zovirax | 1 | 3 |
| Example 1 | 0.05 | 0.03 |
| Example 2 | 0.2 | 0.4 |
| Example 3 | 0.05 | 0.01 |
| Example 4 | 0.75 | 0.75 |
| Example 5 | 0.1 | 0.06 |

Here, IC₅₀ is the half-maximal fluorescence intensity with respect to the non-infected cell control (100% value). The IC₅₀ value can also be referenced to a suitable active compound control (see description of the assay: infected cells in the presence of a suitable concentration of a substance having anti-herpes action, such as, for example, Zovirax 20 μM). This active compound control reaches fluorescence intensities of about 85 to 95% with respect to the cell control.

Preference is given to [5-(aminosulphonyl)-1,3-thiazol-2-yl]propanamide and -acetamide derivatives according to the invention whose IC₅₀ (HSV-1 F/Vero) in the in-vitro screening test system described above is preferably below 50 μM, more preferably below 25 μM and very particularly preferably below 10 μM.

The compounds according to the invention are thus useful active compounds for the treatment and prophylaxis of disorders caused by herpes viruses, in particular Herpes simplex viruses. Examples of indication areas which may be mentioned are:

1) treatment and prophylaxis of herpes infections, in particular Herpes simplex infections in patients displaying symptoms such as Herpes labialis, Herpes genitalis, and HSV-related keratitis, encephalitis, pneumonia, hepatitis etc.

2) treatment and prophylaxis of herpes infections, in particular Herpes simplex infections, in patients with a suppressed immune system (for example AIDS patients, cancer patients, patients having a genetic immunodeficiency, transplant patients)

3) treatment and prophylaxis of herpes infections, in particular Herpes simplex infections, in new-born children and infants

4) treatment and prophylaxis of herpes infections, in particular Herpes simplex infections, and in herpes-positive patients, in particular Herpes-simplex-positive patients, for suppressing recurrence (suppression therapy)

In-vivo action

Animals:

6-week-old female mice, BALB/cABom strain were obtained from a commercial breeder (Bomholtgard Breeding and Research Centre Ltd.).

Infection:

The animals were anaesthetized with diethyl ether (Merck) in a sealed glass vessel. 50 μl of a dilution of the virus stock (infection dose 5×10⁴ Pfu) were introduced into the nose of the anaesthetized animals using an Eppendorf pipette. In 90-100% of the animals, this infection dose causes death by a generalized infection with prominent respiratory and central-nervous symptoms on average after 5 to 8 days.

Treatment and assessment:

6 hours after the infection, the animals were treated with doses of 0.1-100 mg/kg of body mass, 3 times per day, at 7 a.m., 2 p.m. and 7 p.m., for a period of 5 days. The substances were pre-dissolved in DMSO and resuspended in tylose/PBS (Hoechst) (final concentration 1.5% DMSO, 0.5% tylose in PBS).

After the last administration, the animals were monitored further and the time of death was determined.

A comparison of the survival curves showed for the compound of Example 3, for example, an ED₉₀ of about 3 mg/kg for HSV-1, where ED₉₀ means that 90% of the animals survive at this dose.

The novel active compounds can be converted in a known manner into the customary formulations, such as tablets, sugar-coated tablets, pills, granules, aerosols, syrups, emulsions, suspensions and solutions, using inert, non-toxic, pharmaceutically suitable carriers and solvents. Here, the therapeutically active compound should in each case be present in a concentration of about 0.5 to 90% by weight of the total mixture, i.e. in amounts which are sufficient to achieve the dosage range indicated.

The formulations are prepared, for example, by extending the active compounds with solvents and/or excipients, if appropriate using emulsifiers and/or dispersants, it being possible, for example, if the diluent used is water, to use, if appropriate, organic solvents as auxiliary solvents.

Administration is carried out in a customary manner, preferably orally, parenterally or topically, in particular perlingually or intravenously.

In the case of parenteral administration, solutions of the active compounds using suitable liquid carrier materials can be employed.

In general, it has proved advantageous in the case of intravenous administration to administer amounts of from approximately 0.001 to 20 mg/kg, preferably approximately 0.01 to 10 mg/kg, of body weight to achieve effective results, and in the case of oral administration the dose is approximately 0.01 to 30 mg/kg, preferably 0.1 to 20 mg/kg, of body weight.

In spite of this, it may be necessary, if appropriate, to depart from the amounts mentioned, namely depending on the body weight or on the type of administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of the administration of relatively large amounts, it may be advisable to divide this into several individual administrations over the course of the day.

If appropriate, it may be useful to combine the compounds according to the invention with other active substances, in particular antiviral active substances.

### STARTING MATERIALS

### Example I

2-Chloro-4-methyl-1,3-thiazole-5-sulphonyl chloride

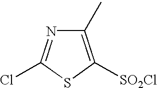

At room temperature, 150 g (1.12 mol) of 2-chloro-4-methyl-1,3-thiazole are added dropwise to a solution of 331 g (2.81 mmol) of thionyl chloride in 653 g (5.61 mmol) of chlorosulphonic acid. The solution is refluxed for 48 h. The mixture is then poured onto 3 l of ice-water and extracted with 4×400 ml of dichloromethane. The combined organic phases are washed with 2.5 l of water, dried over sodium sulphate and concentrated. Distillation of the crude product gives 233.7 g of product in the form of an oil (b.p. 87-96° C., 0.7 mbar, GC 98.1%, yield 89.6%).

### Example II

2-Chloro-4-methyl-1,3-thiazole-5-sulphonamide

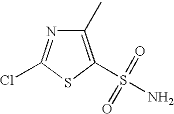

At −10° C., 117.7 g (1.8 mol) of a 26% strength aqueous ammonia solution are added dropwise to a solution of 208 g (95% pure, 0.9 mol) of 2-chloro-4-methyl-1,3-thiazole-5-sulphonyl chloride in 1000 ml of tetrahydrofuran. The mixture is allowed to be stirred without further cooling for 2 h and the reaction mixture is then concentrated using a rotary evaporator. The crude product is used for the next step without further purification.

### Example III

Diethyl 2-[5-(aminosulphonyl)-4-methyl-1,3-thiazol-2(3H)-ylidene]malonate

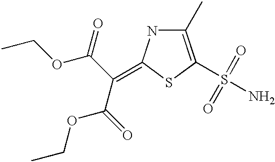

42.2 g (263 mmol) of diethyl malonate are added dropwise to a suspension of 9.74 g (244 mmol) of sodium hydride (60% in mineral oil) in 450 ml of toluene, and the mixture is heated to 95° C. After 5 min, 14 g (66 mmol) of 2-chloro-4-methyl-1,3-thiazole-5-sulphonamide are added dropwise as a saturated solution in toluene, and the mixture is stirred at 95° C. for 96 h. The mixture is then poured into water and the product is extracted with dichloromethane. The combined organic phases are dried over sodium sulphate and concentrated. The crude product is saturated with heptane and the resulting solid is isolated by filtration.

Yield: 14.5 g of a solid (65%).

¹H-NMR (300 MHz, d⁶-DMSO, δ/ppm): 1.25 (m, 6H), 2.5 (s, 3H), 4.15 (m, 4H), 7.85 (s, 2H), 12.6 (s, 1H).

### Example IV

Ethyl 2-[5-(aminosulphonyl)-4-methyl-1,3-thiazol-2(3H)-ylidene]-3-(1,1′-biphenyl-4-ylamino)-3-oxopropanoate

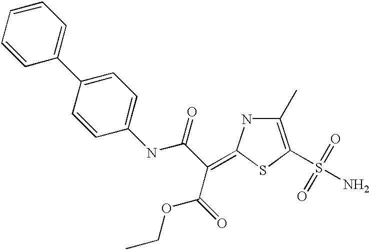

A solution of 5.7 g (16.9 mmol) of diethyl 2-[5-(aminosulphonyl)-4-methyl-1,3-thiazol-2(3H)-ylidene]malonate and 3.4 g (20.3 mmol) of 4-aminobiphenyl in 60 ml of xylene is stirred under reflux for 16 h. The solvent is removed under reduced pressure and the residue is stirred with dichloromethane, whereupon a solid remains. The dichloromethane phase is washed with 1N HCl solution and concentrated. The two solids obtained in this manner are, together, saturated with petroleum ether, and the solid is filtered off with suction.

Yield: 5.7 g (73%)

¹H-NMR (300 MHz, d⁶-DMSO, δ/ppm): 1.4 (t, J=7 Hz, 3H), 2.5 (s, 3H), 4.4 (m, 2H), 7.3-7.9 (m, 11H), 10.3-14.3 (m, 2H). According to the NMR, the product is a mixture of the possible E/Z isomers.

### Example V

Ethyl 2-[5-(aminosulphonyl)-4-methyl-1,3-thiazol-2(3H)-ylidene]-3-oxo-3-{[4-(2-pyridinyl)phenyl]amino}-propanoate

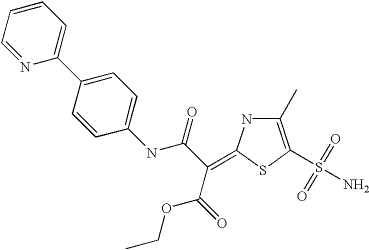

A solution of 2 g (5.95 mmol) of diethyl 2-[5-(aminosulphonyl)-4-methyl-1,3-thiazol-2(3H)-ylidene]malonate and 1.2 g (7.13 mmol) of 4-(2-pyridinyl)-aniline in 20 ml of xylene is stirred under reflux for 16 h. The solvent is removed under reduced pressure and the residue is stirred with dichloromethane, whereupon a solid remains. The dichloromethane phase is washed with 1N HCl solution and concentrated. The two solids obtained in this manner are, together, stirred with petroleum ether, and the solid is filtered off with suction.

Yield: 2.2 g (80%)

¹H-NMR (300 MHz, d⁶-DMSO, δ/ppm): 1.4 (t, J=7 Hz, 3H), 2.5 (s, 3H), 4.4 (m, 2H), 7.7-8.8 (m, 10H), 10.6-14.2 (m, 2H). According to the NMR, the product is a mixture of the possible E/Z isomers.

### Example VI

Ethyl 2-[5-(aminosulphonyl)-4-methyl-1,3-thiazol-2(3H)-ylidene]-3-](2′,5′-difluoro-1,1′-biphenyl-4-yl)amino]-3-oxopropanoate

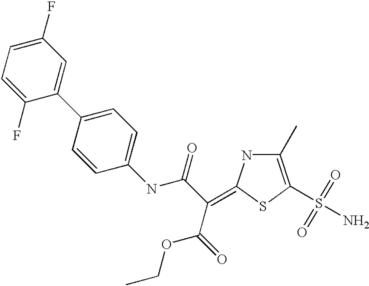

A solution of 1.23 g (3.66 mmol) of diethyl 2-[5-(aminosulphonyl)-4-methyl-1,3-thiazol-2(3H)-ylidene]malonate and 0.9 g (4.39 mmol) of 2′,5′-difluoro-1,1′-biphenyl-4-amine in 12 ml of xylene is stirred under reflux for 16 h. The solvent is removed under reduced pressure and the residue is stirred with dichloromethane, whereupon a solid remains. The dichloromethane phase is washed with 1N HCl solution and concentrated. The two solids obtained in this manner are, together, stirred with petroleum ether, and the solid is filtered off with suction.

Yield: 1.2 g (66%)

¹H-NMR (300 MHz, d⁶-DMSO, δ/ppm): 1.4 (t, J=7 Hz, 3H), 2.5 (s, 3H), 4.4 (m, 2H), 7.2-7.9 (m, 9H), 10.5-14.2 (m, 2H). According to the NMR, the product is a mixture of the possible E/Z isomers. MS (ESI+): 496 (M+H), 991 (2M+H).

### Example VII

Ethyl 2-[5-(aminosulphonyl)-4-methyl-1,3-thiazol-2(3H)-ylidene]-3-[(4-ethoxy-phenyl) aminol]-3-oxopropanoate

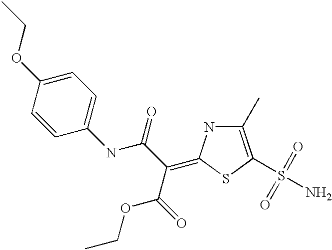

A solution of 5.0 g (14.9 mmol) of diethyl 2-[5-(aminosulphonyl)-4-methyl-1,3-thiazol-2(3H)-ylidene]malonate and 2.45 g (17.8 mmol) of 4-ethoxyaniline in 50 ml of xylene is stirred under reflux for 16 h. The solvent is removed under reduced pressure and the residue is stirred with dichloromethane, whereupon a solid remains. The dichloromethane phase is washed with 1N HCl solution and concentrated. The two solids obtained in this manner are, together, stirred with petroleum ether, and the solid is filtered off with suction.

Yield: 1.2 g (66%)

¹H-NMR (300 MHz, d⁶-DMSO, δ/ppm): 1.3 (t, J=7 Hz, 3H), 1.4 (t, J=7 Hz, 3H), 2.5 (s, 3H), 4.0 (q, J=7 Hz, 2H), 4.4 (m, 2H), 6.9 (d, J=9 Hz, 2H), 7.5 (d, J=9 Hz, 20 2H), 7.8 (broad s, 2H) 10.1-14.4 (m, 2H). According to the NMR, the product is a mixture of the possible E/Z isomers. MS (ESI+): 428 (M+H).

### Example VIII

Ethyl 3-(1,1′-biphenyl-4-ylamino)-2-[4-methyl-5-[(methylamino)sulphonyl]-1,3-thiazol-2(3H)-ylidene]-3-oxopropanoate

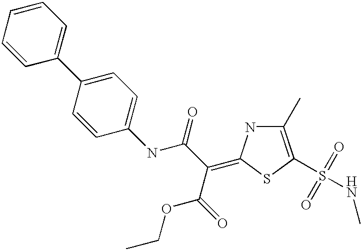

A solution of 3.0 g (8.56 mmol) of diethyl 2-[4-methyl-5-[(methylamino)sulphonyl]-1,3-thiazol-2(3H)-ylidene]malonate and 1.45 g (8.56 mmol) of 4-aminobiphenyl in 30 ml of xylene is stirred under reflux for 16 h. The solvent is removed under reduced pressure and the residue is stirred with dichloromethane, whereupon a solid remains. The dichloromethane phase is washed with 1N HCl solution and concentrated. The two solids obtained in this manner are, together, stirred with petroleum ether, and the solid is filtered off with suction.

Yield: 3.2 g (79%)

¹H-NMR (300 MHz, d⁶-DMSO, δ/ppm): 1.4 (t, J=7 Hz, 3H), 2.5 (s, 3H), 2.55 (d, J=5 Hz, 3H), 4.4 (m, 2H), 7.3-8.0 (m, 10H), 10.4-14.2 (m, 2H). According to the NMR, the product is a mixture of the possible E/Z isomers.

### PREPARATION EXAMPLES

### Example 1

2-[5-(Aminosulphonyl)-4-methyl-1,3-thiazol-2-yl]-N-(1,1′-biphenyl-4-yl)-propanamide

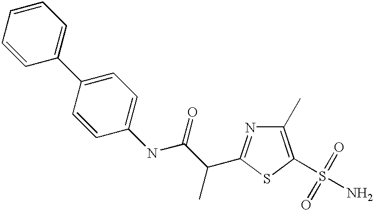

At 0° C., 209 mg (5.22 mmol) of lithium aluminium hydride are introduced a little at a time into a solution of 800 mg (1.74 mmol) of ethyl 2-[5-(aminosulphonyl)-4-methyl-1,3-thiazol-2(3H)-ylidene]-3-(1,1′-biphenyl-4-ylamino)-3-oxopropanoate in 30 ml of tetrahydrofuran. The solution is allowed to warm to room temperature and stirred for another 2 h. The mixture is quenched with water and extracted with dichloromethane. The crude product is purified on a silica gel column (chloroform:methanol 2-20%).

Yield: 120 mg (17%)

¹H-NMR (300 MHz, d⁶-DMSO, δ/ppm): 1.55 (d, J=7 Hz, 3H), 2.5 (s, 3H), 4.3 (q, J=7 Hz, 1H), 7.3-7.8 (m, 11H), 10.5 (s, 1H).

### Example 2

2-[5-(Aminosulphonyl)-4-methyl-1,3-thiazol-2-yl]-N-[4-(2-pyridinyl)-phenyl]propanamide

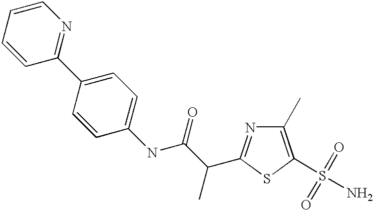

At 0° C., 288 mg (7.6 mmol) of lithium aluminium hydride are introduced a little at a time into a solution of 1000 mg (2.17 mmol) of ethyl 2-[5-(aminosulphonyl)-4-methyl-1,3-thiazol-2(3H)-ylidene]-3-oxo-3-{[4-(2-pyridinyl)phenyl]amino}-propanoate in 40 ml of tetrahydrofuran. The mixture is allowed to warm to room temperature and stirred at 40° C. for another 4 h. The mixture is quenched with water and extracted with dichloromethane. The crude product is purified on a silica gel column (chloroform:methanol 2-20%).

Yield: 510 mg (58%)

¹H-NMR (300 MHz, d⁶-DMSO, δ/ppm): 1.55 (d, J=7 Hz, 3H), 2.5 (s, 3H), 4.3 (q, J=7 Hz, 1H), 7.3-8.7 (m, 10H), 10.6 (s, 1H).

### Example 3

2-[5-(Aminosulphonyl)-4-methyl-1,3-thiazol-2-yl]-N-(2′,5′-difluoro-1,1′-biphenyl-4-yl)propanamide

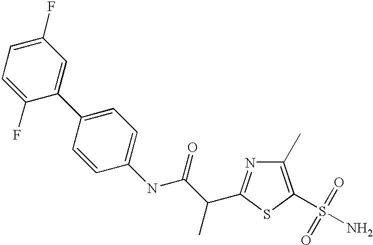

At 0° C., 115 mg (3.03 mmol) of lithium aluminium hydride are introduced a little at a time into a solution of 500 mg (1.01 mmol) of ethyl 2-[5-(aminosulphonyl)-4-methyl-1,3-thiazol-2(3H)-ylidene]-3-[(2′,5′-difluoro-1,1′-biphenyl-4-yl)amino]-3-oxopropanoate in 20 ml of tetrahydrofuran. The solution is allowed to warm to room temperature and stirred at room temperature for another 2 h. The mixture is quenched with water and extracted with dichloromethane. The crude product is purified on a silica gel column (chloroform:methanol 2-20%).

Yield: 50 mg (11%)

¹H-NMR (300 MHz, d⁶-DMSO, δ/ppm): 1.55 (d, J=7 Hz, 3H), 2.5 (s, 3H), 4.3 (q, J=7 Hz, ¹H), 7.2-7.9 (m, 9H), 10.0 (s, 1H). MS(ESI+): 438 (M+H).

### Example 4

2-[5-(Aminosulphonyl)-4-methyl-1,3-thiazol-2-yl]-N-(4-ethoxyphenyl)-propanamide

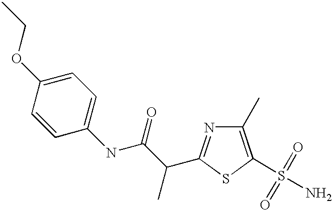

At 0° C., 266 mg (7.02 mmol) of lithium aluminium hydride are introduced a little at a time into a solution of 1000 mg (2.34 mmol) of ethyl 2-[5-(aminosulphonyl)-4-methyl-1,3-thiazol-2(3H)-ylidene]-3-[(4-ethoxyphenyl)amino]-3-oxopropanoate in 40 ml of tetrahydrofuran. The solution is allowed to warm to room temperature and stirred at room temperature for another 2 h. The mixture is quenched with water and extracted with dichloromethane. The crude product is purified on a silica gel column (chloroform: methanol 2-20%).

Yield: 56 mg (6%)

¹H-NMR (300 MHz, CDCl₃, CD₃OD, δ/ppm): 1.4 (t, J=7 Hz, 3H), 1.65 (d, J→7 Hz, 3H), 2.6 (s, 3H), 4.05 (q, J=7 Hz, 2H), 4.1 (q, J=7 Hz, 1H), 6.8 (d, J=9 Hz, 2H), 7.45 (d, J=9 Hz, 2H).

### Example 5

N-(1,1′-Biphenyl-4-yl)-2-{4-methyl-5-[(methylamino)sulphonyl]-1,3-thiazol-2-yl }propanamide

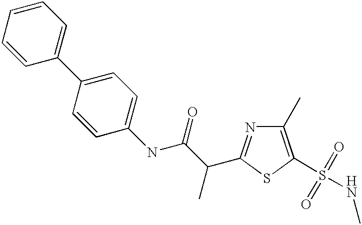

At 0° C., 240 mg (6.33 mmol) of lithium aluminium hydride are introduced a little at a time into a solution of 1000 mg (2.11 mmol) of ethyl 3-(1,1′-biphenyl-4-ylamino)-2-[4-methyl-5-[(methylamino)sulphonyl]-1,3-thiazol-2(3H)-ylidene]-3-oxopropanoate in 40 ml of tetrahydrofuran. The solution is allowed to warm to room temperature and stirred at room temperature for another 6 h. The mixture is quenched with water and extracted with dichloromethane. The crude product is purified on a silica gel column (chloroform:methanol 2-20%).

Yield: 580 mg (66%)

¹H-NMR (300 MHz, d⁶-DMSO, δ/ppm): 1.6 (d, J=7 Hz, 3H), 2.5 (m, 6H), 4.3 (q, J=7 Hz, 1H), 7.3-7.9 (m, 10H), 10.55 (s, 1H). MS(ESI+): 416 (M+H).

### Example 6

N-(1,1′-Biphenyl-4-yl)-2-{4-methyl-5-[(methylamino)sulphonyl]-1,3-thiazol-2-yl}acetamide

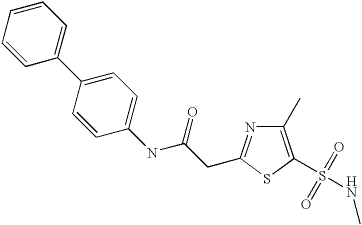

1 l of 1M of lithium hydroxide solution is added to a solution of 100 mg (0.19 mmol) of ethyl 3-(1,1′-biphenyl-4-ylamino)-2-[4-methyl-5-[(methylamino)sulphonyl]-1,3-thiazol-2(3H)-ylidene]-3-oxopropanoate in 2 ml of dimethoxyethane. The mixture is stirred under reflux for 6 h and then concentrated. The crude product is taken up in dichloromethane, washed with water, dried and concentrated using a rotary evaporator. The crude product is purified on a silica gel column (chloroform:methanol 2-20%).

Yield: 56 mg (73%)

H-NMR (300 MHz, d⁶-DMSO, δ/ppm): 2.5 (m, 6H), 4.25 (s, 2H), 7.3-7.9 (m, 10H), 10.5 (s, 1H). MS(ESI+): 402 (M+H).

1. Compounds of the general formula (I): 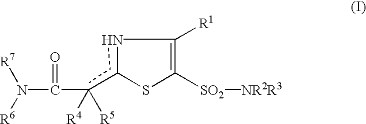
in which
R¹ represents hydrogen, halogen, (C₁-C₆)-alkyl, (C₁-C₆)-alkoxy, amino-(C₁-C₆)-alkyl or halogeno-(C₁-C₆)-alkyl,
R² and R³ are identical or different and represent hydrogen or represent (C₁-C₆)-alkyl which is optionally substituted by 1 to 3 substituents selected from the group consisting of (C₃-C₆)-cycloalkyl, (C₁-C₆)-alkoxy, halogen, hydroxyl, amino and (C₆-C₁₀)-aryl which for its part may be substituted by hydroxyl or (C₁-C₆)-alkoxy, or
R² and R³ together with the nitrogen atom form a 5- or 6-membered saturated heterocycle which may optionally also contain an oxygen atom,
R⁴ represents hydrogen, (C₁-C₆)-acyl, (C₂-C₆)-alkenyl, (C₃-C₈)-cycloalkyl or (C₁-C₆)-alkoxycarbonyl, or
R⁴ represents (C₁-C₆)-alkyl which may optionally be substituted by 1 to 3 substituents selected from the group consisting of halogen, hydroxyl, (C₁-C₆)-acyl, (C₁-C₆)-alkoxy, —(OCH₂CH₂)ₙOCH₂CH₃, where n is 0 or 1, phenoxy, (C₆-C₁₀)-aryl and —NR¹³R¹⁴,
where
R¹³ and R¹⁴ are identical or different and represent hydrogen, (C₁-C₆)-acyl, (C₁-C₆)-alkyl, carbamoyl, mono- or di-(C₁-C₆)-alkylamino-(C₁-C₆)alkyl, mono- or di-(C₁-C₆)-alkyl-aminocarbonyl, (C₆-C₁₀)-aryl or (C₁-C₆)-alkoxycarbonyl, or
R¹³ and R¹⁴ together with the nitrogen atom form a 5- or 6-membered saturated heterocycle which may optionally contain a further heteroatom from the group consisting of S and O or a radical of the formula —NR¹⁵ and which may be substituted by oxo,
where
R¹⁵ represents hydrogen or (C₁-C₄)-alkyl,
R⁵ represents no substituent, hydrogen, (C₁-C₆)-alkyl or (C₁-C₆)-alkanoyl,
R⁶ represents phenyl which may optionally be substituted by one to three substituents selected from the group consisting of
halogen,
(C₆-C₁₀)-aryl which may optionally be substituted by 1 to 3 substituents selected from the group consisting of (C₁-C₆)-alkanoyl, (C₁-C₆)-alkoxy, (C -C₆)-alkyl, halogen, (C -C₆)-alkoxycarbonyl, nitro, halogeno-(C₁-C₆)-alkyl, halogeno-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C6)-alkoxycarbonylamino, (C₁-C₆)-alkylsulphoxy, (C₁-C₆)-alkylsulphonyl, tri-(C₁-C₆)-alkylsilyloxy, a 3- to 8-membered saturated or unsaturated non-aromatic mono- or bicyclic heterocycle having up to 3 heteroatoms from the group consisting of S, N and O, which is optionally attached via a nitrogen atom, and cyano,
(C₁-C₆)-alkoxycarbonyl,
(C₁-C₆)-alkylthio,
hydroxyl,
carboxyl,
partially fluorinated (C₁-C₆)-alkoxy having up to 6 fluorine atoms,
(C₁-C₆)-alkyl which may optionally be substituted by a radical of the formula 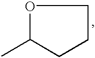
an aromatic heterocycle having up to 3 hetero atoms from the group consisting of S, N and O, which is optionally attached via a nitrogen atom and may optionally be substituted by 1to 3 substituents selected from the group consisting of (C₁-C₆)-alkanoyl, (C₁-C₆)-alkoxy, (C₁-C₆)-alkyl, halogen, (C₁-C₆)-alkoxycarbonyl, nitro, halogeno-(C₁-C₆)-alkyl, halogeno-(C₁-C₆)-alkoxy, amino, (C₁-C₆)-alkylthio, hydroxyl, carboxyl, carbamoyl, mono- or di-(C₁-C₆)-alkylaminocarbonyl, mono-or di-(C₁-C₆)-alkanoylamino, (C₁-C₆)-alkoxycarbonyl amino, (C₁-C₆)-alkylsulphoxy, (C₁-C₆)-alkylsulphonyl, a 3- to 8-membered saturated or unsaturated non-aromatic mono- or bicyclic heterocycle having up to 3 heteroatoms from the group consisting of S, N and O, which is optionally attached via a nitrogen atom, and cyano,
a 3- to 8-membered saturated or unsaturated non-aromatic mono- or bicyclic heterocycle having up to 3 heteroatoms from the group consisting of S, N and O, which is optionally attached via a nitrogen atom and which may optionally be substituted by 1 to 3 substituents selected from the group consisting of oxo, halogen, hydroxyl, (C₁-C₆)-alkoxycarbonyl, (C₁-C₆)-alkoxycarbonylamino, (C₁-C₆)-alkyl, halogeno-(C₁-C₆)-alkyl and hydroxy-(C₁-C₆)-alkyl,
and groups of the formulae
—OR¹⁹,
—NR²⁰R²¹ or —CO—NR²²R²³,
in which
R¹⁹ is phenyl which for its part is optionally substituted by a group of the formula —NR²⁴R²⁵,
in which
R²⁴ and R²⁵ are identical or different and represent hydrogen, (C₁-C₆)-alkyl or (C₁-C₆)-acyl,
or
R¹⁹ represents (C₁-C₆)-alkyl which is optionally mono- to trisubstituted by hydroxyl and/or halogen,
R²⁰ and R ²¹ are identical or different and hydrogen, carbamoyl, mono- or di-(C₁-C₆)-alkylaminocarbonyl, phenyl, (C₁-C₆)-acyl or (C₁-C₆)-alkyl, where the (C₁-C₆)-alkyl mentioned above is optionally substituted by (C₁-C₆)-alkoxy, (C₁-C₆)-acyl, by phenyl or by a 5- or 6-membered aromatic heterocycle having up to 3 heteroatoms from the group consisting of S, N and O, where the phenyl mentioned above and the aromatic heterocycle mentioned above are optionally mono- to trisubstituted by identical or different substituents from the group consisting of halogen and hydroxyl, and
R²² and R²³ are identical or different and represent hydrogen or (C₁-C₆)-alkyl,
R⁷ represents hydrogen, (C₁-C₆)-alkyl or (C₁-C₆)-alkanoyl,
R⁸ represents no substituent, hydrogen or (C₁-C₆)-alkyl, and [symbol]
represents a single or double bond,
and their salts.

2. Compounds of the general formula (I) according to claim 1, in which R¹ represents hydrogen or (C₁-C₆)-alkyl.

3. Compounds of the general formula (I) according to claim 1 or 2, in which R² and R³ each independently represent hydrogen or (C₁-C₆)-alkyl.

4. Compounds of the general formula (I) according to any of claims 1, 2 or 3, in which R⁴ represents hydrogen or methyl.

5. Compounds of the general formula (I) according to any of claims 1, 2, 3 or 4, in which R⁵ represents hydrogen.

6. Compounds of the general formula (I) according to any of claims 1, 2, 3, 4 or 5, in which R⁶ represents phenyl which may optionally be substituted by one to three substituents selected from the group consisting of
(C₆-C₁₀)-aryl which may optionally be substituted by 1 to 3 substituents selected from halogen, and
a 6-membered aromatic heterocycle having 1 heteroatom from the group consisting of S, N and O, which may optionally be substituted by 1 or 2 halogen atoms,
(C₁-C₆)-alkoxy.

7. Compounds of the general formula (I) according to any of claims 1, 2, 3, 4, 5 or 6, in which R⁸ represents hydrogen or no substituent.

8. Compounds according to claim 1 of the following formula: 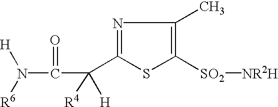
in which
R², R⁴ and R⁶ are as defined in claim 1, and their salts.

9. Compound according to claim 1 of the formula: 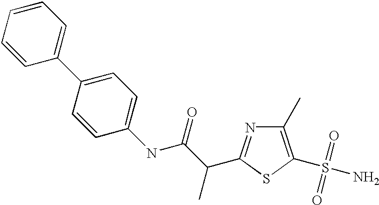
and its pharmaceutically acceptable salts.

10. Compound according to claim 1 of the formula: 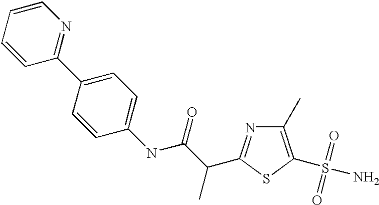
and its pharmaceutically acceptable salts.

11. Compound according to claim 1 of the formula: 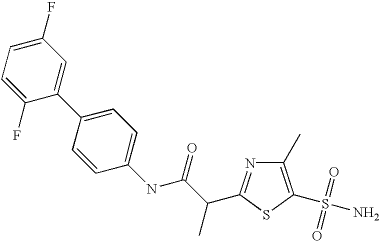
and its pharmaceutically acceptable salts.

12. Compound according to claim 1 of the formula: 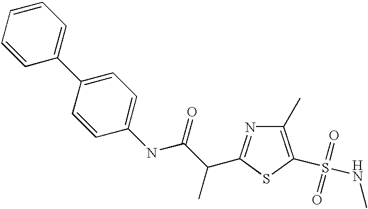
and its pharmaceutically acceptable salts.

13. Compound according to claim 1 of the formula: 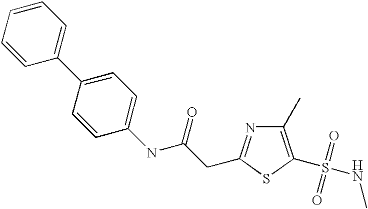
and its pharmaceutically acceptable salts.

14. Process for preparing compounds of the general formula (I) according to claim 1, characterized in that
compounds of the general formula (I′) 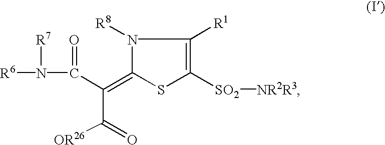
in which
R¹, R², R³, R⁶, R⁷ and R⁸ are as defined above and R²⁶ represents (C₁-C₆)-alkyl, are,
[A] if R⁴ is to be methyl, reacted with lithium aluminium hydride in inert solvents to give compounds of the general formula (I″) 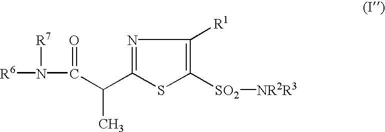
in which
R¹, R², R³ R⁶ and R⁷ are as defined above, or
[B] if R⁴ is to be hydrogen, reacted with lithium hydroxide in inert solvents to give compounds of the general formula (I′″) 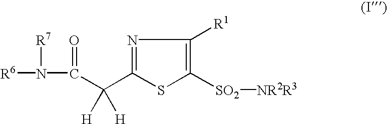
in which
R¹, R², R³, R⁶ and R7 are as defined above,
or
[C] if R⁴ is not to be hydrogen or methyl, reacted with compounds of the general formula (II)
Y-R⁴ (II)
in which
Y represents a leaving group, such as, for example, triflate or halogen, preferably chlorine, and R⁴ is as defined above,
in inert solvents to give compounds of the general formula (III) 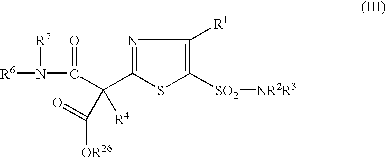
in which
R¹, R², R³, R⁴, R⁶, R⁷ and R²⁶ are as defined above,
and these are then decarboxylated to give compounds of the general formula (I″41 ) 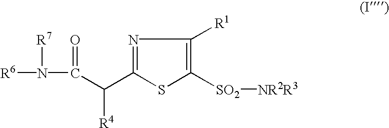
in which
R¹, R², R³, R⁴, R⁶ and R⁷ are as defined above,
and, if R⁵ is a substituent different from hydrogen,
compounds of the general formula (I″) or (I″″)
are reacted with compounds of the general formula (IV)
X-R⁵ (IV)
in which
X represents a leaving group, such as, for example, triflate or halogen, preferably chlorine, and R⁵ is as defined above, in inert solvents, if appropriate in the presence of a base and/or an auxiliary, to give compounds of the formula (I).

15. Compounds according to claim 1 for use as medicaments.

16. Pharmaceutical composition, comprising a compound of the general formula (I) according to claim 1 in a mixture with a pharmaceutically acceptable carrier or excipient.

17. Use of a compound of the general formula (I) according to claim 1 for preparing a medicament.

18. Use of a compound of the general formula (I) according to claim 1 for preparing a medicament for the treatment of viral infections.

19. Use of a compound of the general formula (I) according to claim 1 for preparing a medicament for the treatment of viral infections by herpes viruses.

20. Use of a compound of the general formula (I) according to claim 1 for preparing a medicament for the treatment of viral infections by Herpes simplex viruses.

21. Use of [5-(aminosulphonyl)-1,3-thiazol-2-yl]propanamide derivatives for preparing medicaments.

22. Use of [5-(aminosulphonyl)-1,3-thiazol-2-yl]acetamide derivatives for preparing medicaments.

23. Compounds of the general formula (X) 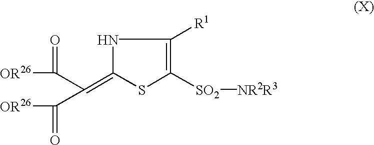
in which
R¹, R², R³ and R²⁶ are as defined above.
